(19) United States
(12) Patent Application Publication
(10) Pub. No.: US 2025/0032670 A1
(43) Pub. Date: Jan. 30, 2025

(54) 3D PRINTED BONE DEFECT REPAIR SCAFFOLD LOADED WITH EXTRACELLULAR MATRIX OF MESENCHYMAL STEM CELLS AND PREPARATION METHOD THEREOF

(71) Applicant: School and Hospital of Stomatology, Guangzhou Medical University, Guangzhou (CN)

(72) Inventors: Qianzhou Jiang, Guangzhou (CN); Xinran Tu, Guangzhou (CN); Lvhua Guo, Guangzhou (CN); Guozhong Tan, Guangzhou (CN); Rongfeng Chen, Guangzhou (CN); Yang Zhang, Guangzhou (CN)

(21) Appl. No.: 18/896,954

(22) Filed: Sep. 26, 2024

Related U.S. Application Data
(63) Continuation of application No. PCT/CN2023/082528, filed on Mar. 20, 2023.

(30) Foreign Application Priority Data
Mar. 23, 2022 (CN) 2022102929271

(51) Int. Cl. A61L 27/36; A61L 27/12; A61L 27/22; A61L 27/54; A61L 27/58

(52) U.S. Cl. CPC A61L 27/3633; A61L 27/12; A61L 27/222; A61L 27/3683; A61L 27/3691; A61L 27/54; A61L 27/58; A61L 2300/252; A61L 2300/414; A61L 2430/02; A61L 2430/40

(57) ABSTRACT

A 3D printed bone defect repair scaffold loaded with extracellular matrix of mesenchymal stem cells is provided, its preparation method includes: dissolving gelatin, sodium alginate and 58S bioglass in water to obtain a solution, stirring the solution evenly to obtain 3D printing slurry, and performing 3D printing; chemically cross-linking the semi-finished scaffold with a calcium chloride solution, followed by soaking in a glutaraldehyde solution to chemically cross-link, and cleaning and freeze-drying to obtain the 3D printed scaffold; sterilizing the 3D printed scaffold; seeding rBMSC suspension on the sterilized 3D printed scaffold at a concentration of at least 10⁶ cells/well, and culturing rBMSC by using a low glucose DMEM with 10% FBS for 2 weeks; and taking out the scaffold to perform a decellularization treatment, and freeze-drying the decellularized 3D printed scaffold to obtain the 3D printed bone defect repair scaffold loaded with extracellular matrix of mesenchymal stem cells.

### TECHNICAL FIELD

The disclosure relates to the field of bone tissue engineering repair and reconstruction technologies, and more particularly to a three-dimension (3D) printed bone defect repair scaffold loaded with extracellular matrix of mesenchymal stem cells and a preparation method thereof.

### BACKGROUND

With the increase of bone tissue damage caused by aging, joint degenerative diseases and trauma such as traffic accidents, more and more attention has been paid to bone defect repair. Bone grafting methods such as autogenous bone grafting, allogenous bone grafting and artificial bone grafting are generally used in clinical practice. The autologous bone grafting is a “gold standard” for defect repair, but the source of autologous bone is limited and often in short supply. The allogeneic bone grafting has a risk of infectious diseases, while the artificial bone grafting lacks osteoinductive activity, has poor osteogenic efficiency (i.e., bone formation efficiency), and is difficult to form new tissues with structures similar to healthy bone tissue. Therefore, the research on new regenerative bone defect repair materials with high biological activity and efficient osteogenesis has become a difficult and hot spot in recent years, and has huge clinical needs and market prospects.

At present, bone defect repair scaffolds are divided into synthetic materials and biological materials according to materials. The synthetic materials have control advantages in terms of strength and configuration, but compatibility and degradability in living organisms need to be improved. The biological materials have good compatibility and degradability, but strength and shape of the biological materials are difficult to meet the requirements. In addition, a scaffold with a three-dimensional structure is more conducive to cell differentiation and proliferation.

Bioglass is an important scaffold material for bone tissue engineering, which can effectively promote biomineralization in vivo, release silicon ions and calcium ions to promote stem cell osteogenesis and angiogenesis. Gelatin or sodium alginate hydrogel is a mixture of natural polymer materials, which has advantages of good biocompatibility, tissue absorbability and low immunogenicity. Especially, the gelatin or the sodium alginate hydrogel is beneficial to combine with highly bioactive inorganic powders to perform 3D printing, but mechanical strength of the gelatin or the sodium alginate hydrogel is relatively poor. The 3D printing can effectively construct a porous bioglass bone tissue engineering scaffold, precisely control parameters such as porosity and pore size, and endow the porous bioglass bone tissue engineering scaffold with better biological activity. The bone defect repair scaffolds usually need to have good mechanical properties, biocompatibility, osteoconductivity and osteoinductivity.

On the basis of composite scaffolds, in order to further improve the osteogenic efficiency of the scaffold, loading growth-promoting factors or drugs on the scaffold has become another effective means to improve bone tissue repair. Specifically, the application of the growth-promoting factors in artificially synthesized composite bioscaffolds has aroused great interest of researchers due to efficient promotion of cell proliferation and differentiation and formation of functional proteins in living organisms. Adding the growth-promoting factors such as bone morphogenetic protein 2 (BMP-2) to the composite scaffold can promote osteogenic differentiation of stem cells. However, due to a short half-life of the growth-promoting factors in vivo, it is necessary to add a large amount of the growth-promoting factors to the scaffold to maintain an effective dose for a long time, which exceeds a safe standard dose of 1.5 milligrams per milliliter (mg/mL), thus causing a series of adverse reactions, such as inflammation, ectopic bone and tumor. Therefore, how to further improve the formation efficiency of the bone tissue without causing the adverse reactions has become an urgent problem to be solved. Studies have shown that extracellular matrix (ECM) of rat bone marrow mesenchymal stem cells (rBMSC) can not only provide necessary protein factors for osteogenic differentiation, but also facilitate formation of the bone tissue due to its special “cell sheet” structure. The disclosure can provide theoretical guidance and experimental data for developing new and effective jaw bone defect repair materials, and provide new explorations for clinical research and development of bone defect substitute materials.

### SUMMARY

A purpose of the disclosure is to provide a 3D printed bone defect repair scaffold loaded with extracellular matrix of mesenchymal stem cells, which uses gelatin, sodium alginate and 58S bioglass and adjusts a content ratio of the gelatin, the sodium alginate and the 58S bioglass, so that the prepared scaffold has high strength, good compatibility and good degradation effect in vivo, and rBMSC can adhere and proliferate on the scaffold, so as to significantly improve the osteogenesis efficiency of bone tissue.

In order to achieve the above purpose, technical solutions provided by the disclosure are as follows.

A 3D printed bone defect repair scaffold loaded with extracellular matrix of mesenchymal stem cells, includes: a 3D printed scaffold and an extracellular matrix of mesenchymal stem cells loaded on the 3D printed scaffold.

A preparation method of the 3D printed bone defect repair scaffold loaded with extracellular matrix of mesenchymal stem cells, includes:
S1, preparing the 3D printed scaffold, including:
   S11, dissolving gelatin, sodium alginate and 58S bioglass in water to obtain a solution; where a concentration of the gelatin in the solution by weight to volume is 18%, a concentration of the sodium alginate in the solution by weight to volume is 5%, and a concentration of the 58S bioglass in the solution by weight to volume is 5.5%;
   S12, stirring the solution evenly to obtain a 3D printing slurry, and performing 3D printing by using the 3D printing slurry; where the performing 3D printing includes: printing 4-8 layers by using a nozzle with a diameter of 0.40 millimeters (mm) to 0.50 mm with a first layer including multiple parallel lines, a second layer including multiple parallel lines perpendicularly connected to upper surfaces of the multiple parallel lines of the first layer, a third layer including multiple parallel lines perpendicularly connected to upper surfaces of the multiple parallel lines of the second layer, and so on; and each new layer includes multiple parallel lines that are perpendicularly connected to the upper surfaces of parallel lines of the previous layer;
   S13, obtaining a semi-finished scaffold after the printing, chemically cross-linking the semi-finished scaffold with a calcium chloride solution for 0.5 h to obtain a first cross-linked scaffold, and soaking the first cross-linked scaffold in a glutaraldehyde solution to chemically cross-link for 6 h to obtain a second cross-linked scaffold; and cleaning and freeze-drying the second cross-linked scaffold to obtain the 3D printed scaffold;
S2, sterilizing the 3D printed scaffold to obtain a sterilized 3D printed scaffold;
S3, seeding rBMSC suspension on the sterilized 3D printed scaffold at a concentration of at least 10⁶ cells/well, and culturing rBMSC by using a low glucose Dulbecco's modified eagle medium (DMEM) with 10% fetal bovine serum (FBS) on the sterilized 3D printed scaffold for 2 weeks, and changing culture medium once every 3 days during the culturing, to thereby obtain a 3D printed scaffold with cultured rBMSC; and
S4, taking out the 3D printed scaffold with cultured rBMSC to perform a decellularization treatment to obtain a decellularized 3D printed scaffold, and freeze-drying the decellularized 3D printed scaffold to obtain the 3D printed bone defect repair scaffold loaded with extracellular matrix of mesenchymal stem cells.

A scaffold material used for bone defect repair must meet the following conditions: 1. biodegradation of the scaffold material does not produce toxic substances; 2. the scaffold material may provide good mechanical support for new tissue; 3. a degradation speed of the scaffold material matches a regeneration speed of the tissue; 4. the scaffold material has pores that allow dispersion of nutrients and metabolites; and 5. an anti-compressive property of the scaffold material matches with that of normal cartilage. In the disclosure, the sodium alginate gel has a three-dimensional culture structure suitable for nutrient exchange of cells, and can maintain a specific shape formed by large surface area and many pores. The gelatin is added to improve a mechanical strength of the sodium alginate gel, simulate an internal environment required for growth of the cells, and has good biocompatibility. The 58S bioglass can stably release silicon (Si) ions, calcium (Ca) ions and the like in the scaffold, stimulate osteoprogenitor cells and promote the growth of new bones at a genetic level.

The content selection of the gelatin, the sodium alginate and the 58S bioglass has a great influence on the overall performance of the scaffold. The high content of the gelatin can obtain good biocompatibility, the sodium alginate has good consolidation forming ability, and the 58S bioglass promotes bone growth. Therefore, how to adjust the amount of the three substances (i.e., the gelatin, the sodium alginate and the 58S bioglass) to obtain a scaffold with good biocompatibility, cell adhesion, biodegradability and bioactive factor loading capacity requires a large number of experimental works. After a large number of experimental research, the inventor selected a weight/volume concentration of each component to be 18% gelatin, 5% sodium alginate, and 5.5% 58S bioglass. In the case of hardly affecting the osteogenic efficiency, the proportion of the 58S bioglass in the 3D printing slurry is reduced to improve smoothness of filaments during printing, so that the printed scaffold structure is more regular, and porosity and spacing are more qualified. The structural pores of the 3D printed scaffold of the disclosure are straight, due to the regular structure, there are no obvious obstacles, and there is no strong fluid resistance in hydrodynamics, which is conducive to infiltration of nutrients and cells into the scaffold, and accelerates the osteogenic efficiency during the repair process.

Specifically, a chemical structure of alginate is shown as follows:

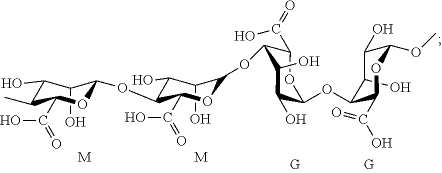

a mechanism of ionic interaction between alginate and divalent cations is as follows:

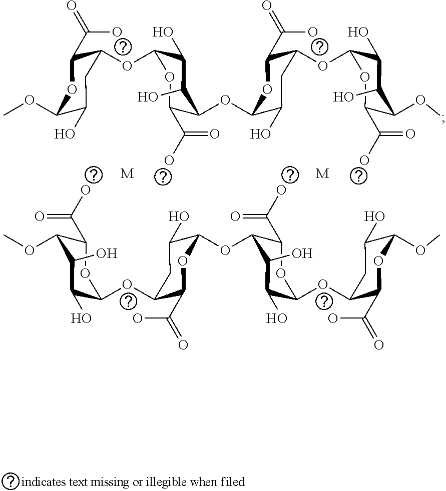

a crosslinking reaction of gelatin with glutaraldehyde is as follows:

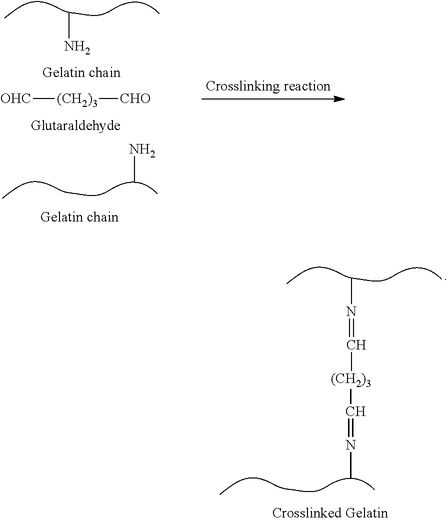

The ECM is a general term for a series of proteins and other components secreted by cells, and plays a key role in cell signal transduction and regulation of cell physiological functions. The ECM of rBMSC can not only provide necessary protein factors for osteogenic differentiation, but also facilitate the formation of the bone tissue due to its special “cell sheet” structure. The 3D printed scaffold of the disclosure can load the ECM of the rBMSC, and the rBMSC can adhere and proliferate on the 3D printed scaffold.

In an embodiment of the disclosure, in the step S12, the performing the 3D printing specifically includes: performing the 3D printing by using a nozzle with a diameter of 0.41 mm at a printing speed of 8 millimeters per second (mm/s), under an air pressure of 0.42 megapascals (Mpa), and at a temperature of 30 Celsius degrees (° C.). The settings of the above printing parameters can maintain a configuration in the forming of the scaffold, meanwhile, the size is more accurate and regular, and there will be no adhesion. Specifically, in the step S12, the solution is evenly stirred by magnetic stirring and/or mechanical stirring to obtain the 3D printing slurry, and the 3D printing slurry is input into a 3D printing material cylinder to perform the 3D printing after defoaming and homogenization.

In an embodiment of the disclosure, in the step S12, a distance between adjacent lines of the multiple parallel lines in each layer is in a range of 300 microns (μm) to 500 μm, and a number of layers of the 3D printed scaffold is 6. The settings of the vertical stacking for the scaffold and the adjustment of the pore distance enhance the diversity and porosity of the scaffold, which is conducive to the infiltration of nutrients and cells into the 3D printed scaffold.

In an embodiment of the disclosure, the 58S bioglass is ground and sieved to obtain 58S bioglass powder, a particle diameter of the 58S bioglass powder is in a range of 4 μm to 10 μm, and a chemical composition of the 58S bioglass is 58% silicon dioxide (SiO₂), 33% calcium oxide (CaO) and 9% phosphorus oxide (P₂O₅). The selection of the diameter of the 58S bioglass powder makes it have a large specific surface area and releases more ions. When the diameter of the 58S bioglass is less than 4 μm, dispersion uniformity of the 58S bioglass in the solution becomes poor, which is not conducive to its efficacy.

In an embodiment of the disclosure, in the step S3, the changing culture medium once every 3 days during the culturing specifically includes: removing the culture medium from a culture dish by using a pipette, rinsing the rBMSC with phosphate buffered saline (PBS) for 3 times, and adding fresh culture medium into the culture dish. In the step S4, the decellularization treatment includes: soaking the 3D printed scaffold with cultured rBMSC in a solution of 10 millimolar per liter (mM) ammonia (also referred to as ammonia solution or ammonia water) and 0.1% sodium dodecyl sulfate (SDS) for 30 minutes (min), followed by rinsing with distilled water for 3 times, soaking in 0.1% deoxyribonuclease (DNase) solution for 10 min, and rinsing with the distilled water for 3 times.

In an embodiment of the disclosure, in the step S4, the freeze-drying decellularized 3D printed scaffold to obtain the 3D printed bone defect repair scaffold loaded with extracellular matrix of mesenchymal stem cells includes: storing the decellularized 3D printed scaffold at −40° C. for 12 hours (h).

The changing the culture medium, the decellularization treatment and the freeze-drying of the 3D printed bone defect repair scaffold loaded with extracellular matrix of mesenchymal stem cells of the disclosure can use conventional technical means in the related art. The above parameters are set as an optimized scheme, which can make the scaffold load more extracellular matrix of mesenchymal stem cells, and the loading rate is high.

In an embodiment of the disclosure, a concentration of the calcium chloride solution is in a range of 5% to 6%, and the calcium chloride solution is obtained by dissolving calcium chloride powder in distilled water. A concentration of the glutaraldehyde solution is in a range of 1.0% to 1.5%, and the glutaraldehyde solution is obtained by diluting 50% glutaraldehyde solution with distilled water. The selections of the concentrations of the calcium chloride solution and the glutaraldehyde solution make a cross-linking effect of the 3D printed scaffold better. Compared to the related art, beneficial effects of the disclosure are as follows.

1. After a large number of experimental research, the disclosure selects the weight/volume concentrations of components to be 18% gelatin, 5% sodium alginate, and 5.5% 58S bioglass. In the case of hardly affecting the osteogenesis efficiency, the 58S bioglass component in the 3D printing slurry is reduced to improve smoothness of filaments during printing, so that the printed scaffold structure is more regular, and porosity and spacing are more qualified. The structural pores of the 3D printed scaffold of the disclosure are straight, due to the regular structure, there are no obvious obstacles, and there is no strong fluid resistance in hydrodynamics, which is conducive to infiltration of nutrients and cells into the scaffold, and accelerates the osteogenic efficiency during the repair process.

2. The disclosure has found through research that the gelatin/sodium alginate/58S bioglass scaffold loaded with the ECM of the rBMSC is applied to bone defect repair, and areas and a number of branches of the formed vascular tissue are significantly improved, which effectively promotes the formation of the bone tissue and the vascular tissue, significantly improves the efficiency of the bone defect repair.

3. The disclosure studies and optimizes preparation process parameters of the gelatin/sodium alginate/58S bioglass scaffold loaded with the ECM of the rBMSC to obtain a good loading effect.

### BRIEF DESCRIPTION OF DRAWINGS

FIG. 1A illustrates a front photo of a 3D printed bone defect repair scaffold loaded with extracellular matrix of mesenchymal stem cells according to an embodiment of the disclosure.

FIG. 1B illustrate a side photo of the 3D printed bone defect repair scaffold loaded with extracellular matrix of mesenchymal stem cells according to an embodiment of the disclosure.

FIG. 2 illustrates a schematic diagram of a live/dead staining result of the 3D printed bone defect repair scaffold loaded with extracellular matrix of mesenchymal stem cells according to an embodiment of the disclosure. Specifically, FIG. 2 shows that rBMSC can adhere and proliferate on the 3D printed bone defect repair scaffold loaded with extracellular matrix of mesenchymal stem cells.

FIG. 3 illustrates a schematic diagram of a surface micro-topography of the 3D printed bone defect repair scaffold loaded with extracellular matrix of mesenchymal stem cells and an adhesion and extension situation of the rBMSC on the 3D printed bone defect repair scaffold loaded with extracellular matrix of mesenchymal stem cells observed by a scanning electron microscope (SEM).

FIG. 4A illustrates a schematic diagram of an expression level of an osteogenic related gene runt-related transcription factor 2 (RUNX-2) in the 3D printed bone defect repair scaffold loaded with extracellular matrix of mesenchymal stem cells.

FIG. 4B illustrates a schematic diagram of an expression level of an osteogenic related gene BMP-2 in the 3D printed bone defect repair scaffold loaded with extracellular matrix of mesenchymal stem cells.

FIG. 5A illustrates a schematic diagram of osteogenic efficiency of a3D printed bone defect repair scaffold loaded with extracellular matrix of mesenchymal stem cells group in an animal experiment.

FIG. 5B illustrates a schematic diagram of osteogenic efficiency of a blank group in the animal experiment.

FIG. 5C illustrates a schematic diagram of osteogenic efficiency of a bone implant ossification substitute (BIO-OSS) bone powder positive control group in the animal experiment.

FIG. 6A illustrates a schematic diagram from a top-down perspective of the 3D printed bone defect repair scaffold loaded with extracellular matrix of mesenchymal stem cells according to an embodiment of the disclosure.

FIG. 6B illustrates a schematic diagram from a side perspective of the 3D printed bone defect repair scaffold loaded with extracellular matrix of mesenchymal stem cells according to an embodiment of the disclosure.

FIG. 6C illustrates a layout diagram of the 3D printed bone defect repair scaffold loaded with extracellular matrix of mesenchymal stem cells according to an embodiment of the disclosure.

### DETAILED DESCRIPTION OF EMBODIMENTS

In order to make purposes, technical solutions and advantages of the disclosure clearer and more understandable, the disclosure is further described in details below in conjunction with drawings and embodiments of the specification. However, a scope of protection required by the disclosure is not limited by the embodiments.

Unless otherwise specified, raw materials used in the following embodiments are commercially available.

Specifically, a chemical composition of the used 58S bioglass includes 58% SiO₂, 33% CaO and 9% P₂O₅, and a diameter of the 58S bioglass is in a range of 4 μm to 10 μm.

### Embodiment 1

A 3D printed bone defect repair scaffold loaded with extracellular matrix of mesenchymal stem cells, includes a 3D printed scaffold and an extracellular matrix of mesenchymal stem cells loaded on the 3D printed scaffold.

A preparation method of the 3D printed bone defect repair scaffold loaded with extracellular matrix of mesenchymal stem cells includes the following steps S1-S4.

In step S1, the 3D printed scaffold is prepared by the following steps S11-S13.

In step S11, gelatin, sodium alginate and 58S bioglass are dissolved in water to obtain a solution. Specifically, weight/volume concentrations of the gelatin, the sodium alginate and the 58S bioglass in the solution are 18%, 5% and 5.5% respectively.

In step S12, the solution is stirred evenly by magnetic stirring and/or mechanical stirring to obtain 3D printing slurry. The 3D printing slurry is input into a 3D printing material cylinder to preform 3D printing after defoaming and homogenization. A nozzle with a diameter of 0.41 mm is used for the 3D printing, and 4 layers are printed at a printing speed of 8 mm/s, under an air pressure of 0.42 Mpa, and at a temperature of 30° C., with a first layer including multiple parallel lines, a second layer including multiple parallel lines perpendicularly connected to upper surfaces of the multiple parallel lines of the first layer, a third layer including multiple parallel lines perpendicularly connected to upper surfaces of the multiple parallel lines of the second layer, and so on. Specifically, each new layer includes multiple parallel lines that are perpendicularly connected to the upper surfaces of parallel lines of the previous layer.

In step S13, a semi-finished scaffold is obtained, and the semi-finished scaffold is chemically cross-linked with a calcium chloride solution for 0.5 h to obtain a first cross-linked scaffold. The first cross-linked scaffold is soaked in a glutaraldehyde solution to chemically cross-link for 6 h to obtain a second cross-linked scaffold. The second cross-linked scaffold is cleaned and freeze-dried to obtain the 3D printed scaffold. A concentration of the calcium chloride solution is 5%, and the calcium chloride solution is obtained by dissolving calcium chloride powder in distilled water. A concentration of the glutaraldehyde solution is 1.0%, and the glutaraldehyde solution is obtained by diluting 50% glutaraldehyde solution with distilled water.

In step S2, the 3D printed scaffold is sterilized to obtain a sterilized 3D printed scaffold.

In step S3, a rBMSC suspension is seeded on the sterilized 3D printed scaffold at a concentration of at least 10⁶ cells/well, and rBMSC cells are cultured on the sterilized 3D printed scaffold by using a low glucose DMEM with 10% FBS for 2 weeks. Culture medium is changed once every 3 days during the culturing. A specific process of changing the culture medium in the step S3 includes the following steps: the culture medium is removed from a culture dish by using a pipette, the rBMSC cells are rinsed with PBS for 3 times, and fresh culture medium is added into the culture dish.

In step S4, the 3D printed scaffold with cultured rBMSC is taken out to perform a decellularization treatment by soaking the 3D printed scaffold with cultured rBMSC in a solution of 10 mM ammonia and 0.1% SDS for 30 min, followed by rinsing with distilled water for 3 times, soaking in 0.1% DNase solution for 10 min, and rinsing with the distilled water for 3 times to obtain a decellularized 3D printed scaffold. The decellularized 3D printed scaffold is stored at −40° C. for 12 h to obtain the 3D printed bone defect repair scaffold loaded with extracellular matrix of mesenchymal stem cells.

### Embodiment 2

A 3D printed bone defect repair scaffold loaded with extracellular matrix of mesenchymal stem cells, includes a 3D printed scaffold and an extracellular matrix of mesenchymal stem cells loaded on the 3D printed scaffold.

A preparation method of the 3D printed bone defect repair scaffold loaded with extracellular matrix of mesenchymal stem cells includes the following steps S1-S4.

In step S1, the 3D printed scaffold is prepared by the following steps S11-S13.

In step S11, gelatin, sodium alginate and 58S bioglass are dissolved in water to obtain a solution. Specifically, weight/volume concentrations of the gelatin, the sodium alginate and the 58S bioglass in the solution are 18%, 5% and 5.5% respectively.

In step S12, the solution is stirred evenly by magnetic stirring and/or mechanical stirring to obtain 3D printing slurry. The 3D printing slurry is input into a 3D printing material cylinder to preform 3D printing after defoaming and homogenization. A nozzle with a diameter of 0.41 mm is used for the 3D printing, and 6 layers are printed at a printing speed of 8 mm/s, under an air pressure of 0.42 Mpa, and at a temperature of 30° C., with a first layer including multiple parallel lines, a second layer including multiple parallel lines perpendicularly connected to upper surfaces of the multiple parallel lines of the first layer, a third layer including multiple parallel lines perpendicularly connected to upper surfaces of the multiple parallel lines of the second layer, and so on. Specifically, each new layer includes multiple parallel lines that are perpendicularly connected to the upper surfaces of parallel lines of the previous layer.

In step S13, a semi-finished scaffold is obtained, and the semi-finished scaffold is chemically cross-linked with a calcium chloride solution for 0.5 h to obtain a first cross-linked scaffold. The first cross-linked scaffold is soaked in a glutaraldehyde solution to chemically cross-link for 6 h to obtain a second cross-linked scaffold. The second cross-linked scaffold is cleaned and freeze-dried to obtain the 3D printed scaffold. A concentration of the calcium chloride solution is 5.5%, and the calcium chloride solution is obtained by dissolving calcium chloride powder in distilled water. A concentration of the glutaraldehyde solution is 1.5%, and the glutaraldehyde solution is obtained by diluting 50% glutaraldehyde solution with distilled water.

In step S2, the 3D printed scaffold is sterilized to obtain a sterilized 3D printed scaffold.

In step S3, a rBMSC suspension is seeded on the sterilized 3D printed scaffold at a concentration of at least 10⁶ cells/well, and rBMSC cells are cultured on the sterilized 3D printed scaffold by using a low glucose DMEM with 10% FBS for 2 weeks. Culture medium is changed once every 3 days during the culturing. A specific process of changing the culture medium in the step S3 includes the following steps: the culture medium is removed from a culture dish by using a pipette, the rBMSC cells are rinsed with PBS for 3 times, and fresh culture medium is added into the culture dish.

In step S4, the 3D printed scaffold with cultured rBMSC is taken out to perform a decellularization treatment by soaking the 3D printed scaffold with cultured rBMSC in a solution of 10 mM ammonia and 0.1% SDS for 30 min, followed by rinsing with distilled water for 3 times, soaking in 0.1% DNase solution for 10 min, and rinsing with the distilled water for 3 times to obtain a decellularized 3D printed scaffold. The decellularized 3D printed scaffold is stored at −40° C. for 12 h to obtain the 3D printed bone defect repair scaffold loaded with extracellular matrix of mesenchymal stem cells.

### Embodiment 3

A 3D printed bone defect repair scaffold loaded with extracellular matrix of mesenchymal stem cells, includes a 3D printed scaffold and an extracellular matrix of mesenchymal stem cells loaded on the 3D printed scaffold.

A preparation method of the 3D printed bone defect repair scaffold loaded with extracellular matrix of mesenchymal stem cells includes the following steps S1-S4.

In step S1, the 3D printed scaffold is prepared by the following steps S11-S13.

In step S11, gelatin, sodium alginate and 58S bioglass are dissolved in water to obtain a solution. Specifically, weight/volume concentrations of the gelatin, the sodium alginate and the 58S bioglass in the solution are 18%, 5% and 5.5% respectively.

In step S12, the solution is stirred evenly by magnetic stirring and/or mechanical stirring to obtain 3D printing slurry. The 3D printing slurry is input into a 3D printing material cylinder to preform 3D printing after defoaming and homogenization. A nozzle with a diameter of 0.41 mm is used for the 3D printing, and 8 layers are printed at a printing speed of 8 mm/s, under an air pressure of 0.42 Mpa, and at a temperature of 30° C., with a first layer including multiple parallel lines, a second layer including multiple parallel lines perpendicularly connected to upper surfaces of the multiple parallel lines of the first layer, a third layer including multiple parallel lines perpendicularly connected to upper surfaces of the multiple parallel lines of the second layer, and so on. Specifically, each new layer includes multiple parallel lines that are perpendicularly connected to the upper surfaces of parallel lines of the previous layer.

In step S13, a semi-finished scaffold is obtained, and the semi-finished scaffold is chemically cross-linked with a calcium chloride solution for 0.5 h to obtain a first cross-linked scaffold. The first cross-linked scaffold is soaked in a glutaraldehyde solution to chemically cross-link for 6 h to obtain a second cross-linked scaffold. The second cross-linked scaffold is cleaned and freeze-dried to obtain the 3D printed scaffold. A concentration of the calcium chloride solution is 6%, and the calcium chloride solution is obtained by dissolving calcium chloride powder in distilled water. A concentration of the glutaraldehyde solution is 1.5%, and the glutaraldehyde solution is obtained by diluting 50% glutaraldehyde solution with distilled water.

In step S2, the 3D printed scaffold is sterilized to obtain a sterilized 3D printed scaffold.

In step S3, a rBMSC suspension is seeded on the sterilized 3D printed scaffold at a concentration of at least 10⁶ cells/well, and rBMSC cells are cultured on the sterilized 3D printed scaffold by using a low glucose DMEM with 10% FBS for 2 weeks. Culture medium is changed once every 3 days during the culturing. A specific process of changing the culture medium in the step S3 includes the following steps: the culture medium is removed from a culture dish by using a pipette, the rBMSC cells are rinsed with PBS for 3 times, and fresh culture medium is added into the culture dish.

In step S4, the 3D printed scaffold with cultured rBMSC is taken out to perform a decellularization treatment by soaking the 3D printed scaffold with cultured rBMSC in a solution of 10 mM ammonia and 0.1% SDS for 30 min, followed by rinsing with distilled water for 3 times, soaking in 0.1% DNase solution for 10 min, and rinsing with the distilled water for 3 times to obtain a decellularized 3D printed scaffold. The decellularized 3D printed scaffold is stored at −40° C. for 12 h to obtain the 3D printed bone defect repair scaffold loaded with extracellular matrix of mesenchymal stem cells.

### Performance Testing

The performance testing is performed on the 3D printed bone defect repair scaffold loaded with extracellular matrix of mesenchymal stem cells prepared in the embodiment 2, and a specific testing process is as follows.

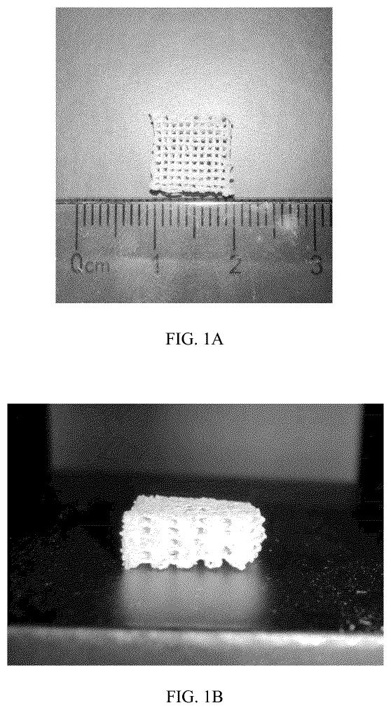

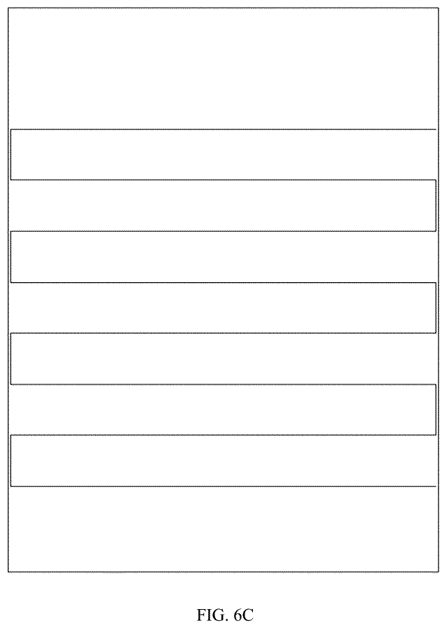

1. The 3D printed bone defect repair scaffold loaded with extracellular matrix of mesenchymal stem cells prepared in the embodiment 2 is photographed, and a structural size of the 3D printed bone defect repair scaffold loaded with extracellular matrix of mesenchymal stem cells is shown in FIGS. 1A and 1B and FIGS. 6A and 6B.

2. Polymerase chain reaction (PCR) test: rBMSC cells are seeded at a density of 10⁵ cells/well onto the 3D printed bone defect repair scaffold loaded with extracellular matrix of mesenchymal stem cells, the rBMSC cells are cultured in the low glucose DMEM with 10% FES, the culture medium is changed once every 3 days, and total RNA of the rBMSC cells is extracted at days 0, 7, and 14 to perform the PCR test.

### 3. Animal Experiment

64 specific pathogen free (SPF) level male Sprague-Dawley (SD) rats with weight of 280 grams (g) to 320 g are randomly divided into an ECM scaffold group (i.e., a 3D printed bone defect repair scaffold loaded with extracellular matrix of mesenchymal stem cell group), a BIO-OSS collagen group (i.e., a BIO-OSS bone powder positive control group) and a control group (i.e., a blank group). Each rat is intraperitoneally injected for anaesthetization under sterile conditions, and a 1.0 centimeter (cm) to 1.5 cm incision is made on a lower edge parallel to a mandible of the rat. The mandible of each rat is exposed by blunt dissection after subcutaneous tissue is cut in layers, and a ring drill with a diameter of 5 mm is combined with physiological saline infusion cooling to prepare a circular full-thickness bone defect with a diameter of 5 mm. The scaffold loaded with ECM and BIO-OSS collagen are implanted on the rats respectively, and the blank group is not placed with materials. The wound in the tissue of each rat is sutured in layers with 5-0 sutures, and penicillin sodium is intramuscularly injected to each rat for 3 consecutive days after surgery to combat infection. Samples are collected at two time points of 4 weeks and 8 weeks: the rats are euthanized by carbon dioxide asphyxiation, the mandible of the rat including the defect area is removed and fixed in 10% neutral buffered formalin for 24 h, and then scanned by micro-computed tomography (Micro-CT). A NRECON software SKYSCAN is used to scan and reconstruct the image files, and a region of interest (ROI) is selected from the scanned images for analysis.

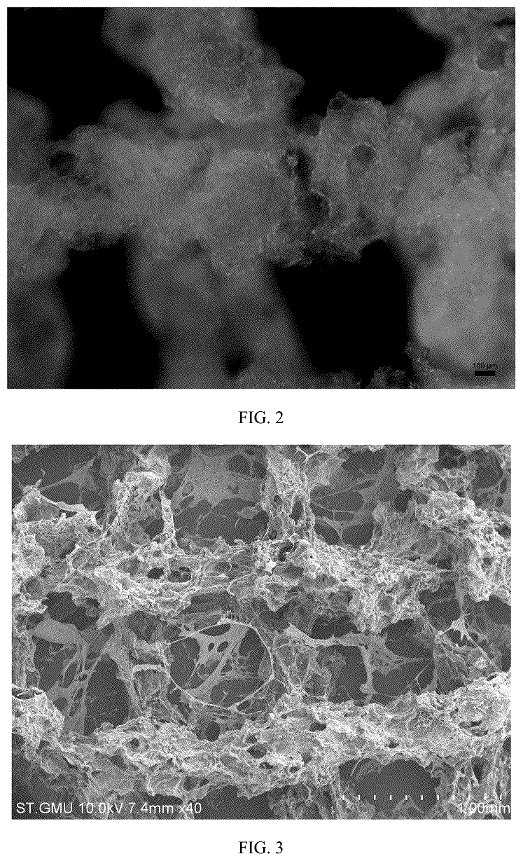

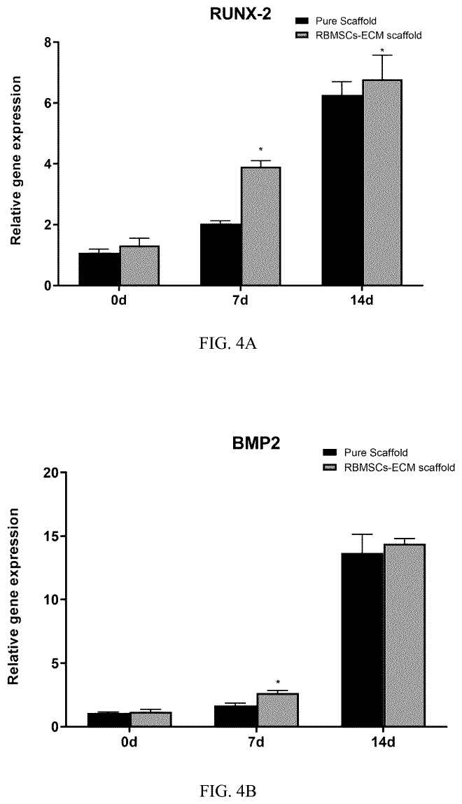

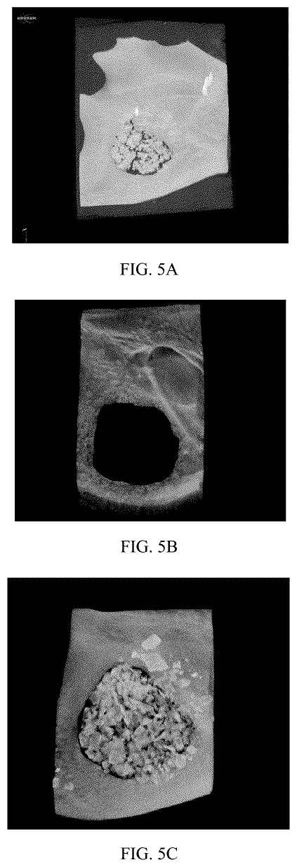

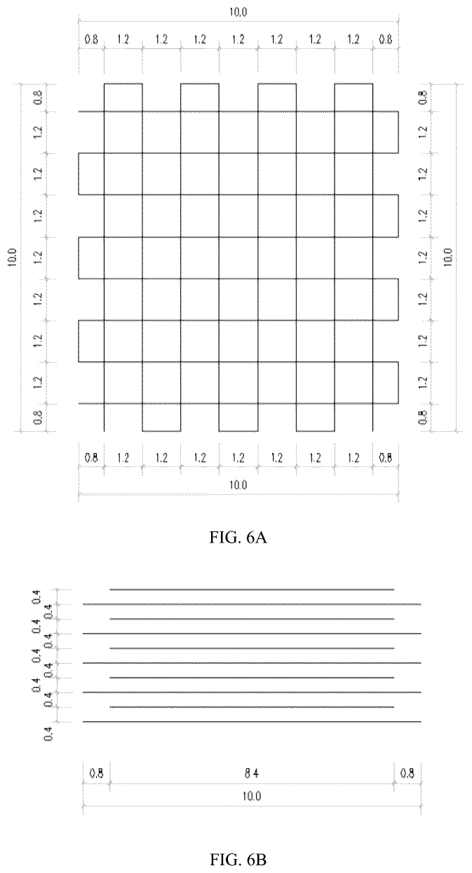

It can be seen from FIGS. 1A and 1B that the structural pores of the 3D printed scaffold of the disclosure are straight, the regular structure lacks obvious obstacles and exhibits minimal fluid resistance in hydrodynamics, facilitating the infiltration of nutrients and cells into the scaffold, and accelerating the osteogenic efficiency during the repair process. It can be seen from FIG. 2 and FIG. 3 that the rBMSC cells can adhere to and proliferate on the 3D printed bone defect repair scaffold loaded with extracellular matrix of mesenchymal stem cells. It can be seen from FIGS. 4A and 4B that the expression levels of osteogenic related genes in the 3D printed bone defect repair scaffold loaded with extracellular matrix of mesenchymal stem cells are high. It can be seen from FIGS. 5A-5C that osteogenic efficiency of the 3D printed bone defect repair scaffold loaded with extracellular matrix of mesenchymal stem cells is comparable to that of the BIO-OSS bone powder (i.e., the BIO-OSS collagen).

It can be seen from the above testing results that the 3D printed bone defect repair scaffold loaded with extracellular matrix of mesenchymal stem cells prepared by the disclosure achieves the purpose of the disclosure, which has good loading effect. When applied for bone defect repair, the obtained 3D printed bone defect repair scaffold can effectively promote the formation of the bone tissue and the vascular tissue, thereby significantly improving the efficiency of the bone defect repair.

According to the revelation and teaching of the above-mentioned specification, those skilled in the art can also make changes and amendments to the above-mentioned embodiment. Therefore, the disclosure is not limited to the specific embodiments disclosed and described above, and some amendments and changes to the disclosure should also fall within the scope of protection of the claims of the disclosure. In addition, although some specific terms are used in the specification, these terms are merely for convenience of description and do not constitute any limitation to the disclosure.

What is claimed is:

1. A three-dimension (3D) printed bone defect repair scaffold loaded with extracellular matrix of mesenchymal stem cells, comprising:
a 3D printed scaffold; and
an extracellular matrix of mesenchymal stem cells loaded on the 3D printed scaffold;
wherein a preparation method of the 3D printed bone defect repair scaffold loaded with extracellular matrix of mesenchymal stem cells comprises:
S1, preparing the 3D printed scaffold, comprising:
S11, dissolving gelatin, sodium alginate and 58S bioglass in water to obtain a solution; wherein a concentration of the gelatin in the solution by weight to volume is 18%, a concentration of the sodium alginate in the solution by weight to volume is 5%, and a concentration of the 58S bioglass in the solution by weight to volume is 5.5%;
S12, stirring the solution evenly to obtain a 3D printing slurry, and performing 3D printing by using the 3D printing slurry; wherein the performing 3D printing comprises: printing 4-8 layers by using a nozzle with a diameter of 0.40 mm to 0.50 mm with a first layer comprising a plurality of parallel lines, a second layer comprising a plurality of parallel lines perpendicularly connected to upper surfaces of the plurality of parallel lines of the first layer, a third layer comprising a plurality of parallel lines perpendicularly connected to upper surfaces of the plurality of parallel lines of the second layer, and so on;
S13, obtaining a semi-finished scaffold after the printing, chemically cross-linking the semi-finished scaffold with a calcium chloride solution for 0.5 h to obtain a first cross-linked scaffold, and soaking the first cross-linked scaffold in a glutaraldehyde solution to chemically cross-link for 6 h to obtain a second cross-linked scaffold; and cleaning and freeze-drying the second cross-linked scaffold to obtain the 3D printed scaffold;
S2, sterilizing the 3D printed scaffold to obtain a sterilized 3D printed scaffold;
S3, seeding a rat bone marrow mesenchymal stem cell (rBMSC) suspension on the sterilized 3D printed scaffold at a concentration of at least 10⁶ cells/well, and culturing rBMSC by using a low glucose Dulbecco's modified eagle medium (DMEM) with 10% fetal bovine serum (FBS) on the sterilized 3D printed scaffold for 2 weeks, and changing culture medium once every 3 days during the culturing, to thereby obtain a 3D printed scaffold with cultured rBMSC; and
S4, taking out the 3D printed scaffold with cultured rBMSC to perform a decellularization treatment to obtain a decellularized 3D printed scaffold, and freeze-drying the decellularized 3D printed scaffold to obtain the 3D printed bone defect repair scaffold loaded with extracellular matrix of mesenchymal stem cells.

2. The 3D printed bone defect repair scaffold loaded with extracellular matrix of mesenchymal stem cells as claimed in claim 1, wherein in the step S12, the performing 3D printing specifically comprises: performing the 3D printing by using a nozzle with a diameter of 0.41 mm, at a printing speed of 8 mm/s, under an air pressure of 0.42 Mpa, and at a temperature of 30° C.

3. The 3D printed bone defect repair scaffold loaded with extracellular matrix of mesenchymal stem cells as claimed in claim 1, wherein in the step S12, a distance between adjacent lines of the plurality of parallel lines in each layer is in a range of 300 μm to 500 μm, and a number of layers of the 3D printed scaffold is 6.

4. The 3D printed bone defect repair scaffold loaded with extracellular matrix of mesenchymal stem cells as claimed in claim 1, wherein the 58S bioglass is ground and sieved to obtain 58S bioglass powder, a particle diameter of the 58S bioglass powder is in a range of 4 μm to 10 μm, and a chemical composition of the 58S bioglass is 58% silicon dioxide (SiO₂), 33% calcium oxide (CaO) and 9% phosphorus oxide (P₂O₅).

5. The 3D printed bone defect repair scaffold loaded with extracellular matrix of mesenchymal stem cells as claimed in claim 1, wherein in the step S3, the changing culture medium once every 3 days during the culturing specifically comprises: removing the culture medium from a culture dish by using a pipette, rinsing the rBMSC with phosphate buffered saline (PBS) for 3 times, and adding fresh culture medium into the culture dish; and in the step S4, the decellularization treatment comprises: soaking the 3D printed scaffold with cultured rBMSC in a solution of 10 mM ammonia and 0.1% sodium dodecyl sulfate (SDS) for 30 min, followed by rinsing with distilled water for 3 times, soaking in 0.1% deoxyribonuclease (DNase) solution for 10 min, and rinsing with the distilled water for 3 times.

6. The 3D printed bone defect repair scaffold loaded with extracellular matrix of mesenchymal stem cells as claimed in claim 1, wherein in the step S4, the freeze-drying decellularized 3D printed scaffold to obtain the 3D printed bone defect repair scaffold loaded with extracellular matrix of mesenchymal stem cells comprises: storing the decellularized 3D printed scaffold at −40° C. for 12 h.

7. The 3D printed bone defect repair scaffold loaded with extracellular matrix of mesenchymal stem cells as claimed in claim 1, wherein a concentration of the calcium chloride solution is in a range of 5% to 6%, and the calcium chloride solution is obtained by dissolving calcium chloride powder in distilled water.

8. The 3D printed bone defect repair scaffold loaded with extracellular matrix of mesenchymal stem cells as claimed in claim 1, wherein a concentration of the glutaraldehyde solution is in a range of 1.0% to 1.5%, and the glutaraldehyde solution is obtained by diluting 50% glutaraldehyde solution with distilled water.

9. The 3D printed bone defect repair scaffold loaded with extracellular matrix of mesenchymal stem cells as claimed in claim 1, wherein in the step S12, the performing 3D printing by using the 3D printing slurry, further comprises: inputting the 3D printing slurry into a 3D printing material cylinder to performing the 3D printing after defoaming and homogenization.

10. The 3D printed bone defect repair scaffold loaded with extracellular matrix of mesenchymal stem cells as claimed in claim 1, wherein in the step S12, the stirring the solution evenly to obtain 3D printing slurry, comprises: stirring the solution evenly by at least one of magnetic stirring and mechanical stirring to obtain the 3D printing slurry.
